(19) United States
(12) Patent Application Publication
(10) Pub. No.: US 2005/0008685 A1
(43) Pub. Date: Jan. 13, 2005

(54) Oral rehydration compositions containing liposomes

(76) Inventors: Cheryl R. Mitchell, Stockton, CA (US); James B. Mitchell, Manteca, CA (US)

Correspondence Address: The Halvorson Law Firm, Ste 1, 405 W. Southern Ave., Tempe, AZ, 85282, US

(21) Appl. No.: 10/614,940

(22) Filed: Jul. 7, 2003

(51) Int. Cl. A61K 9/127; A61K 39/00; A61K 31/70

(52) U.S. Cl. 424/450; 514/2; 514/23

(57) ABSTRACT

A rehydration composition and oral delivery system is provided that allows for enhanced functional ingredient delivery when ingested orally as a water based solution. The rehydration composition comprises a liposomed electrolyte composition comprised of sodium, potassium, citrate, and/or bicarbonate. The rehydration composition, which is concentrated or dried, becomes an oral rehydration solution (ORS) when mixed with water for oral consumption. The rehydration composition, may optionally contain carbohydrates, or functional ingredients, liposomed or not, such as vaccines, drugs, amino acids, mineral salts, vitamins, nutraceuticals, probiotics, prebiotics, flavors, or nutritive or non-nutritive sweeteners. These oral rehydration compositions may then be concentrated to greater than 78% solids or dried to less than 5% moisture. The composition may also be concentrated to less than 78% solids and packaged aseptically or stabilized with preservative agents.

### FIELD OF THE INVENTION

The present invention relates to the field of rehydration and buffer compositions comprised of electrolytes. More specifically, the present invention relates to dried, or dehydrated, rehydration and buffer compositions, that, when blended with water, are suitable for oral ingestion, comprising as an ingredient, at least one liposomal preparation.

### BACKGROUND

Oral Rehydration Compositions containing water, electrolytes, and carbohydrates have been successfully utilized for the treatment of dehydration. Dehydration is the loss of water and body salts (electrolytes) through sweat or illnesses that result in diarrhea or vomiting. Successful replacement of the electrolytes and water, which have been lost by the body, is called rehydration.

It has been recognized that water alone was not effective in treating dehydration. Diluting the blood with water could rapidly result in hyponatremia, a serious condition where the blood has a low sodium level. To prevent hyponatremia, salts containing electrolytes, such as sodium, potassium, and citrate, must be an essential feature of any rehydration composition. However, salts not taken in balance with the water may not be absorbed at the time needed, and could result in hypernatremia or a high sodium level in the blood. This condition is equally as damaging and is very hard on the kidneys. Balanced water and salt solutions, besides being difficult to consume because of taste, may not always be absorbed by the body. Normal digestion as well as certain disease states can interfere with the intestinal mechanism that would normally absorb salts.

More recently, it was discovered that other nutrients, such as amino acids and carbohydrates, could promote the absorption of electrolytes via an alternative mechanism. Carbohydrates such as glucose provide the additional benefit of sufficient sweetness to promote organoleptic acceptability of the salt product. Oral rehydration compositions containing glucose in combination with electrolytes, when dissolved in water, produce an oral rehydration solution “ORS”, which has had a significant impact on the survival rate of cholera victims.

Different carbohydrates such as glucose, sucrose, and complex carbohydrates sources have been utilized successfully to improve the absorption of the electrolytes via a mechanism that is commonly referred to as “nutrient assisted” absorption. However, it is critical that the osmolarity of the oral rehydration solution never exceeds that of the blood since net absorption is dependent on both the nutrient assisted absorption as well as the osmotic forces. A hypertonic solution will pull fluid from the blood into the gut and decrease the net absorption. This “osmotic effect” is known as the “osmotic penalty”. By contrast, a hypotonic solution will increase net absorption using these same osmotic forces. The object of oral rehydration solutions is to replace fluid and electrolyte loss as quickly as possible, which is best accomplished using a hypotonic solution having the correct electrolytes that match that which is being lost.

Oral rehydration compositions containing complex carbohydrate have been found to be superior to glucose because they provide more glucose molecules without adding to the overall osmolarity. If one were to increase the concentration of glucose in an attempt to increase the amount of absorption, the osmotic penalty would overcome the potential benefit. This benefit is summarized by the concept of “increased” glucose carrying capacity without the osmotic penalty. An additional benefit of the complex carbohydrates is the nutritional and caloric value provided by the carbohydrates which are very beneficial in cases where the rehydration solution may be the only source of calories and energy for the individual.

Buffer compositions containing electrolytes and carbohydrates have also been used as an oral delivery system for drugs or vaccines. These buffer compositions protect the activity or functionality of the drug or vaccine from being destroyed by the strong pH of the stomach. As in the oral rehydration compositions, the presence of carbohydrates assists the absorption of the electrolytes as well as the drug or vaccine material. Up until this invention, the nutrient assisted absorption provided by carbohydrates or amino acids, has been understood and utilized to provide an alternative mechanism for the absorption of electrolytes.

Liposomes are currently used as a carrier for targeted drug delivery (delivering drugs where they are needed). The concept is that by surrounding hydrophilic molecules with hydrophobic molecules, agents otherwise impermeable to the cell membranes might be escorted inside the cell, with potential advantages of targeting drugs to an intracellular location. The basic component of all clinically useful liposomes is the phospholipid molecule. Phospholipids are amphiphilic molecules composed of a polar hydrophilic head group and two hydrophobic fatty acid chains attached to a three-carbon glycerol backbone. When phospholipids are mixed with water, they spontaneously rearrange into concentric bilayer structures, termed liposomes or vesicles, separated by aqueous compartments. Liposomes are either unilamellar or multilamellar spheres that are constructed using a variety of lipids.

Stability of the liposomes depends upon the nature of the constituent phospholipid molecules used to construct them, as well as the composition and concentration of the inclusion material. Pure phospholipid bilayers undergo a transition from a gel to a lipid crystalline state at a narrow range of temperature around a characteristic transition temperature (T_{c}). The T_{c} of a given lipid is dependent upon the carbon chain length, the degree of unsaturation, and the nature of the polar head group. Liposome bilayers become more permeable at or above their Tc, and thus release their internal contents. A wide spectrum of phospholipids with a variety of T_{c} can, alone or in combination with sterols, provide a range of membrane fluidity, which in turn controls the permeability of the membranes, and bilayer stability.

Various pharmacological agents of varying solubility and size (anti-tumour and antimicrobial agents, enzymes, peptides, hormones, vaccines, and genetic materials) have already been encapsulated in either the aqueous or the lipid phase of the liposomes. Proteins and other non-lipid molecules can be incorporated into the lipid membranes. A high percent of inclusion (greater than 70%) of the target composition within the liposome has been very critical in the case of drug delivery. Liposomal preparations having stability and a high inclusion percentage significantly affects the method of preparation and subsequently the cost, limiting the cost-effective usage of liposomes to the pharmacetuical industry.

### PRIOR ART

Some prior art teaches the use of other forms of carbohydrates in oral rehydration compositions. U.S. Pat. No. 5,096,894, by Tao et al., U.S. Pat. No. 5,489,440, by Ndife et al., and U.S. Pat. No. 5,120,539, by Lebenthal et al, all teach the use of complex carbohydrates from rice from soluble dextrins to gelatinized starch. All are apparently effective in providing the nutrient assisted absorption of the electrolytes in oral rehydration compositions.

In recent years, there have been certain cereal-based oral rehydration and buffer compositions that have been made with whole rice or a complex carbohydrates from starch sources. These cereal-based oral rehydration and buffer compositions may contain a minimal concentration of fat or phospholipid naturally present in the cereal grain (less than 0.05% on a solids basis). However, oral rehydration and buffer compositions do not contain fat, oil, or phospholipids as an added ingredient.

Buffer compositions have been used as a delivery system for products that are orally ingested. Some of which are U.S. Pat. Nos. 5,242,802; 4,251,509; 5,352,448; 5,176,909; 4,752,474; 4,622,223; 5,057,411; 4,927,628; 4,661,350; 4,337,314; 4,681,762; 5,364,756; 4,957,736; 5,079,165; 5,000,952; 5,001,225; 5,240,704; 4,920,213; 5,147,646; 5,294,441; 4,152,413. Buffer compositions containing electrolytes and carbohydrates have been described (U.S. Pat. No. 5,741,680) which allows for an optimization of pH level reduction in the stomach while providing a rapid transfer of oral vaccine through the stomach itself.

The functionality of the latter rehydration and buffer compositions is believed to be due to the improved absorption of the electrolytes including other functional ingredients or vaccines, through a mechanism whereby the presence of the carbohydrates assist with the absorption of the electrolytes and other functional ingredients. Notably, all oral rehydration compositions have a significant content of simple sugars such as glucose, fructose, or maltose for the purpose of improving organoleptic acceptance, and, more importantly, for the perceived notion that these simple sugars are essential in oral rehydration solutions because of their carrying power that enables the metabolic absorption of the ions.

### SUMMARY OF THE INVENTION

The present invention provides for oral rehydration and buffer compositions in which a portion of the electrolytes have been encapsulated in a liposome. Additionally, the composition may contain, if desired, any one, or combination thereof, of the following ingredients (liposomed or not) including but not limited to minerals, buffers, drugs, vaccines, carbohydrates, proteins or amino acids, minerals, vitamins, or other functional nutraceuticals. The consequence of including, in an oral rehydration composition, a portion of the electrolytes that have been liposomed, and, optionally other ingredients that have been liposomed, is to provide an alternative mechanism for absorption of the liposomed ingredient, by means other than is provided by the nutrient assist mediated absorption. Additionally these oral rehydration compositions containing the liposomed electrolytes and optional liposomed nutracetuicals have the advantage of having a significantly improved orgnoleptic profile compared to those compositions in which the electrolytes have not been liposomed.

We have discovered that, by incorporating a portion of the electrolyte components of the oral rehydration or buffer compositions into liposomes, several advantages are demonstrated:

1. Physiologically, the liposome adds another mechanism of absorption to that already provided by glucose assisted transport. The latter mechanism is more efficient in providing transport of the electrolytes into the cells. Consequently rehydration proceeds more rapidly and is not dependent upon the functioning of the normal nutrient assist or electrolyte absorption pathways. 2. From the standpoint of organoleptic acceptability, oral rehydration and buffer compositions containing liposomes provide an advantage since compositions containing liposomed salts taste less salty. Solutions containing zinc, magnesium, and other electrolytes, which have flavors that are normally objectionable, are distinctly more organoleptically acceptable.

It is an object of the present invention to provide an oral rehydration or buffer composition, suitable for dilution in water, and comprised of an effective amount of electrolytes, so as to produce an oral rehydration or buffer solution so as when dissolved in water, the resulting solution has an osmolality of less than 400 milliomoles; said composition further comprising a portion of the electrolytes that have first been liposomed using a phospholipid so as to produce liposomes having a particle size of preferably less than 0.5 microns so as to maximize the ability for absorption by the intestinal villus.

It is another object of the present invention to provide an oral rehydration composition, as above, further comprising an effective amount of nutritive or non-nutritive sweeteners to improve organoleptic qualities.

It is a further object of the present invention to provide an oral rehydration composition, as above, further comprising of at least one nutraceutical or at least one functional ingredient.

The novel features that are considered characteristic of the invention are set forth with particularity in the appended claims. The invention itself, however, both as to its structure and its operation together with the additional objects and advantages thereof will best be understood from the following description of the preferred embodiment of the present invention. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF THE PREFERRED EMBODIMENT

It has been found that when electrolytes, such as sodium, potassium, citrate or bicarbonate, were liposomed by first combining with a phospholipid in an aqueous solution and then homogenizing under high pressure or sonication to produce liposome particles of less than 0.5 micron in size and having an inclusion volume containing at least 25% of the electrolyte solution, and included into an oral rehydration or buffer composition, the liposomed electrolytes of said composition could be rapidly absorbed through the intestinal villus. It was a surprising and unexpected result, based on results from a rat perfusion study, that the liposomed electrolytes were not only absorbed rapidly but apparently by a pathway in the digestive system other than the standard electrolyte absorption or by the method of a “nutrient assist” from either carbohydrates, proteins, or amino acids. Additionally, it was found that the organoleptic objections (bad taste) found with electrolyte solutions of similar concentration were significantly reduced by at least 25% as compared with the compositions of the present invention containing liposomes.

It was realized that other ingredient compounds that support the healthy maintenance of the body, generally referred to as “nutraceuticals” or “functional” ingredients, could also be added to the electrolyte base prior to incorporation into-the liposome. Normally, because of the poor taste of high concentrations of electrolytes and nutraceuticals in water solutions without liposoming at least some of the particles, the taste would be too objectionable for commercial acceptance.

In considering that glucose or other carbohydrates were not an essential part of an effective oral rehydration composition and, in fact, discovering that liposomed electrolytes containing virtually no glucose or carbohydrates (less than 10% on a dry substance basis) could be used in an oral rehydration composition to produce an extremely effective ORS, we also discovered that solutions that contained liposomed electrolytes and liposomed nutraceuticals were effective in transporting these electrolytes and nutraceuticals and have a superior effect on rehydration and replacement of fluid loss as well as providing a source of nutraceuticals. Also, because of lower osmolarity resulting from solutions containing only electrolytes or small concentrations of carbohydrates, the new oral rehydration composition, according to the present invention, can additionally act as a carrier or oral delivery system for other compounds, such as vaccines, drugs, amino acids, vitamins, minerals, nutraceuticals, prebiotics and probiotics, and cause their metabolic uptake to be significantly increased.

In the prior art, other oral rehydration compositions and solutions rely upon glucose, maltose and or sucrose as an essential part of the carbohydrate base because of its carrying power for the electrolytes, but also for their function as a sweetening agent to improve the organoleptic acceptability of the product. Unfortunately, these sweetening sources also had a high impact on osmolarity. We found that either nutritive or non-nutritive sweeteners may be used in the composition of this invention and still maintain an osmolarity of less than 300 milliosmoles.

The composition of the present invention containing a liposomed electrolyte can be made with or without carbohydrates and optionally, nutraceutical ingredient agents that may or may not have also been liposomed. By having the freedom to adjust the quantity of carbohydrates that may be used in the composition, the quantity and type of electrolyte, or other nutritional or nutraceutical agent that may be added to the oral rehydration composition may be increased while still maintaining the required low osmolarity of the composition. The liposomed electrolytes or nutraceuticals provide for significant increase in uptake or carrying power for delivery of the vaccines, drugs, amino acids, minerals, vitamins, nutraceuticals, probiotics and/or prebiotics.

We also found that by using the liposomed electrolytes, described in the present invention, in making oral rehydration compositions, that we could, if so desired, also add nutritive sweeteners (sucrose, glucose, fructose, or any combination thereof), non-nutritive sweeteners, flavors, acidics, and/or bodying agents, such as pectin or starches. Previously, the carbohydrates necessary for nutrient assist in the oral rehydration solutions were selected from simple sugars. These simple sugars used in oral rehydration compositions provided an improved organoleptic acceptability of the electrolyte composition, but the amount of simple sugars that were used was limited by constraints in osmolarity of the resulting oral rehydration composition. Later, the use of complex carbohydrates allowed for a significant increase in the total amount of carbohydrates that could be utilized in the composition without exceeding the osmolarity requirement. However, due to the limited sweetness provided by the more complex carbohydrates such as pregelatinized starch or maltodextrins, it was desirable to add either nutritive or non-nutritive sweeteners, along with a wide range of flavoring, and adjunct agents, to the composition and still remain below the critical 300 milliosmoles for the prepared solution.

The use of complex carbohydrates in addition to the liposomed electrolytes enables LIS to use as much as 40 to 50 grams of the carbohydrates, additionally being able to add flavors, nutritive or non-nutritive sweeteners to deliver the electrolytes, and optionally being able to add other ingredients, liposomed or not, such as vaccines, drugs, vitamins, amino acids, minerals, nutraceuticals, probiotics, prebiotics, or other compositionally functional adjunct agents, while further enhancing the quick delivery and effectiveness of the agent being delivered. The oral rehydration composition still has preferably less than 300 milliosmoles upon dilution to the ready to drink oral rehydration solution. While it is preferable to have less than 300 milliosmoles for rehydration solutions, it may be suitable to have up to 400 milliosmoles for those rehydration solutions that are intended to be utilized on a one time basis, for example, in the case of delivering vaccines. Rehydration solutions, which are intended for continuous use or are expected to be consumed on a regular basis, such as to inhibit or diminish diarrhea, or for purposes of sweat replacement, preferably are less than 300 milliosmoles.

The oral rehydration compositions and systems as described above may be further dried to less than 5% moisture, or concentrated to the equivalent of between 76 and 85 Brix, to achieve a format that is microbiologically stable and suitable for packaging without additional processing. Drying to less than 5% moisture may be done by spray, freeze, or drum drying or other suitable drying method that can achieve a moisture of less than 5%. Concentration of the rehydration composition may be achieved by the use of a standard evaporator capable of handling viscous fluids and viscosities in excess of 100 poise at 100° Fahrenheit. These concentrated or dried rehydration compositions and systems, may also be diluted in water to achieve a ready to drink product having a total soluble solids of between of 0.2% to 8.0% and, if desired, further processed in an aseptic system to achieve commercial sterility in any suitable aseptic packaging format. If desired, concentration of the rehydration composition may also be limited to less than 78% solids and packaged aseptically or stabilized with preservative type agents.

The following are specific examples illustrating various uses according to the present invention.

### EXAMPLE 1

A Therapeutic Liposomal Electrolyte Preparation (TLEP) suitable for use in preparations of oral rehydration compositions and solutions for applications where it is desirable to replace electrolytes lost from vomiting or diarrhea.

The TLEC was prepared by making an aqueous solution containing liposomed electrolytes according to the present invention having the electrolyte composition (by weight percent) as follows: potassium chloride (5.3%); sodium chloride (9.29%); trisodium citrate dihydrate (10.42%); phosphatidyl choline (2%); and water (73%). Alternatively, an antioxidant water can be used, which contained 0.14% mixed tocopherol and 0.05% Citric acid to prevent oxidation of the phosphatidyl choline. The TLEC solution was found to have a particle size distribution such that all liposomed particles were less than 0.5 micron. The perceived salty taste of this liposomed electrolyte preparation was diminished by about 75% from an electrolyte preparation that had not been liposomed.

### EXAMPLE 2

The TLEC from Example 1 was further dried to less than 5% moisture to produce a TLEC that could be further dry blended with other ingredients.

### EXAMPLE 3

The TLEC of Example 1 was used to make a Therapeutic Oral Rehydration Composition (TORC) concentrate and a ready to drink Therapeutic Oral Rehydration Solution (TORS) having the following formulation:

| | Ready to Drink | TORC Concentrate |
|---|---|---|
| | TORS | (32% Solids) |
| TLEC | 2.8% | 16.8% |
| Complex carbohydrates* | 4.0% | 24.24% |
| Sucralose, dry | 0.0075% | 0.045% |
| Flavor (Strawberry) | 0.35% | 2.4% |
| Pectin, dry | 0.045% | 0.51% |
| Color (Red 40), dry | 0.0033% | 0.02% |
| Water | 92.7942% | 55.985% |

*Complex carbohydrates were tapioca syrup solids

### EXAMPLE 4

The TORC concentrate of Example 3 was spray dried to 4.5% moisture. 53 grams of this dried composition was then dissolved in sufficient water to produce 1 liter of an oral rehydration solution suitable for ingestion and replacement of electrolytes lost from diarrhea.

### EXAMPLE 5

The TORC concentrate of Example 3 was further concentrated using a vacuum evaporator to produce a syrup having a soluble solids content of 78%. 65 gram of this concentrated syrup was then further dissolved in sufficient water to produce 1 liter of an oral rehydration solution suitable for ingestion and replacement of electrolytes lost from diarrhea.

### EXAMPLE 6

A Sport Liposomal Electrolyte Preparation (SLEP) suitable for use in preparations of oral rehydration composition and solutions in applications where it is desirable to replace electrolytes lost from sweat.

The SLEC was prepared by making an aqueous solution containing liposomed electrolytes having the composition as follows: potassium chloride (4.34%); sodium chloride (11.83%); trisodium citrate dihydrate (8.83%); phosphatidyl choline (2.00%); and water (73%). Alternatively, an antioxidant water can be used, which contains 0.14% mixed tocopherol and 0.05% Citric acid to prevent oxidation of the phosphatidyl choline. The SLEC solution was found to have a particle size distribution such that all liposomed particles were less than 0.5 micron. The perceived salty taste of this liposomed electrolyte preparation was diminished by about 50% from an electrolyte preparation that had not been liposomed.

### EXAMPLE 7

The SLEC of Example 6 was further dried to less than 5% moisture to produce a SLEC that could be further dry blended with other ingredients.

### EXAMPLE 8

The SLEC of Example 6 was then used to make a Sport Oral Rehydration Composition (SORC) or a ready to drink Sport Oral Rehydration Solution (SORS) containing the following formulation:

| | Ready to Drink | SORC Concentrate |
|---|---|---|
| | SORS | (51% Solids) |
| TLEC | 0.70 | 7.80 |
| Complex carbohydrates* | 3.00 | 33.43 |
| Sucrose | 1.00 | 11.14 |
| Sucralose, dry | 0.01 | 0.11 |
| Inulin, dry | 0.045 | 0.50 |
| Citric Acid | 0.180 | 2.00 |
| Water | 95.065 | 45.02 |

*Complex carbohydrates were corn maltodextrin

### EXAMPLE 9

The SORC of Example 6 was spray dried to 3.0% moisture. 46 grams of this dried composition was then dissolved in sufficient water to produce 1 liter of an oral rehydration solution suitable for ingestion and replacement of electrolytes lost from diarrhea.

### EXAMPLE 10

The SORC concentrate of Example 6 was further concentrated using a vacuum evaporator to produce a syrup having a soluble solids content of 80%. 55 gram of this concentrated syrup was then further dissolved in sufficient water to produce 1 liter of an oral rehydration solution suitable for ingestion and replacement of electrolytes lost from diarrhea.

The preferred embodiment of the invention is described above in the Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. It should also be noted that the term “osmolality” which refers to moles per kilogram and the term “osmolarity” which specifies moles per liter, while different terms, are being used in this patent interchangeably due to the low density values and hence low impact on the conversion of osmolaity to osmolarity of the oral rehydration solutions.

1. An oral rehydration composition comprising water and an effective amount of liposomed electrolytes.

2. The oral rehydration composition according to claim 1 having an osmolality of less than 400 milliosmoles.

3. The oral rehydration composition according to claim 1 wherein the liposomed electrolytes are phospholipids having an inclusion volume containing electrolytes.

4. The oral rehydration composition according to claim 2 wherein the liposomed electrolytes are phospholipids having an inclusion volume containing electrolytes.

5. The oral rehydration composition according to claim 1 further comprising at least one additive selected from the group consisting of nutraceuticals, carbohydrates, proteins, and amino acids.

6. The oral rehydration composition according to claim 2 further comprising at least one additive selected from the group consisting of nutraceuticals, carbohydrates, proteins, and amino acids.

7. The oral rehydration composition according to claim 3 further comprising at least one additive selected from the group consisting of nutraceuticals, carbohydrates, proteins, and amino acids.

8. The oral rehydration composition according to claim 3 wherein the inclusion volume of the liposomed electrolytes further comprise at least one additive selected from the group consisting of vaccines, drugs, amino acids, vitamins, minerals, flavors, acidic, bodying agents, nutraceuticals, prebiotics and probiotics.

9. The oral rehydration composition according to claim 4 wherein the inclusion volume of the liposomed electrolytes further comprise at least one additive selected from the group consisting of vaccines, drugs, amino acids, vitamins, minerals, flavors, acidic, bodying agents, nutraceuticals, prebiotics and probiotics.

10. A composition for use as an oral rehydration agent comprising an effective amount of liposomed electrolytes and less than 10% water by weight.

11. The oral rehydration composition according to claim 10 further comprising an effective amount of at least one additive selected from the group consisting of flavors, acidics, bodying agents, nutritive and non-nutritive sweeteners to improve organoleptic qualities.

12. The composition according to claim 10 further comprising at least one additive selected from the group consisting of flavor components, nutraceuticals, carbohydrates, and non-nutritive sweeteners.

13. The composition according to claim 10 wherein the less than 10% water is less than 5% water.

14. The composition according to claim 10 concentrated to a syrup of between 76 and 85 Brix.

15. The composition according to claim 11 wherein the less than 10% water is less than 5% water.

16. The composition according to claim 11 concentrated to a syrup of between 76 and 85 Brix.

17. The composition according to claim 12 wherein the less than 10% water is less than 5% water.

18. The composition according to claim 12 concentrated to a syrup of between 76 and 85 Brix.

19. A method for orally rehydrating a subject comprising the steps of:
a. obtaining an oral rehydration solution comprising water and an effective amount of liposomed electrolytes; and
b. drinking the oral rehydration solution.

20. The method according to claim 19 wherein the liposomed electrolytes further comprise phospholipids having an inclusion volume containing electrolytes.

21. The method according to claim 20 wherein the inclusion volume of the liposomed electrolytes further comprise at least one additive selected from the group consisting of vaccines, drugs, amino acids, vitamins, minerals, flavors, acidic, bodying agents, nutraceuticals, prebiotics and probiotics.

22. The method according to claim 20 wherein the rehydration solution further comprises at least one additive selected from the group consisting of nutraceuticals, carbohydrates, proteins, and amino acids.

23. The method according to claim 21 wherein the rehydration solution further comprises at least one additive selected from the group consisting of nutraceuticals, carbohydrates, proteins, and amino acids.

24. A method for orally rehydrating a subject comprising the steps of:
a. obtaining a dehydrated rehydration composition comprising liposomed electrolytes and less than 10% water by weight;
b. mixing the dehydrated rehydration composition with water; and
c. drinking the resultant rehydration solution.

25. The method according to claim 24 wherein the liposomed electrolytes further comprise phospholipids having an inclusion volume containing electrolytes.

26. The method according to claim 25 wherein the inclusion volume of the liposomed electrolytes further comprise at least one additive selected from the group consisting of vaccines, drugs, amino acids, vitamins, minerals, flavors, acidic, bodying agents, nutraceuticals, prebiotics and probiotics.

27. The method according to claim 25 wherein the rehydration composition further comprises at least one additive selected from the group consisting of nutraceuticals, carbohydrates, proteins, and amino acids.

28. The method according to claim 26 wherein the rehydration composition further comprises at least one additive selected from the group consisting of nutraceuticals, carbohydrates, proteins, and amino acids.
